(19) United States
(12) Patent Application Publication
(10) Pub. No.: US 2009/0163601 A1
(43) Pub. Date: Jun. 25, 2009

(54) (R)-2-METHYL-(2,5-DIMETHYLPHENYL)PROPANOL AND (S)-(2-METHYL-2,5-DIMETHYLPHENYL)PROPANOL

(75) Inventors: Matthew P. Meyer, Modesto, CA (US); Jason West, Atwater, CA (US)

Correspondence Address: FOLEY & LARDNER LLP, 975 PAGE MILL ROAD, PALO ALTO, CA, 94304, US

(73) Assignee: The Regents of the University of California

(21) Appl. No.: 11/963,506

(22) Filed: Dec. 21, 2007

(51) Int. Cl. A61K 31/045

(52) U.S. Cl. 514/730

(57) ABSTRACT

This invention is directed to enantiomerically enriched (R)-2-methyl-(2,5-dimethylphenyl)propanol and (S)-2-methyl-(2,5-dimethylphenyl)propanol. The chiral alcohols are synthesized in enantiomerically enriched form by the reaction of 2-methyl-(2,5-dimethylphenyl)propanone using a chiral oxazaborolidine catalyst.

### FIELD OF THE INVENTION

This invention is directed to enantiomerically enriched (R)-2-methyl-(2,5-dimethylphenyl)propanol and (S)-2-methyl-(2,5-dimethylphenyl)propanol. The chiral alcohols are synthesized in enantiomerically enriched form by the reaction of 2-methyl-(2,5-dimethylphenyl)propanone using a chiral oxazaborolidine catalyst.

### BACKGROUND OF THE INVENTION

The availability of enantiomerically enriched chiral building-blocks is essential for the pharmaceutical industry as most drugs contain stereocenters. Since 1988, the FDA has required that every chiral drug candidate be tested in both enantiomerically pure and racemic form. The use of chiral secondary alcohols for the elaboration to more complex molecules is widespread. Therefore, the synthesis, isolation and characterization of novel chiral building blocks is a prerequisite for the advancement of modern medicine.

### SUMMARY OF THE INVENTION

The compounds disclosed herein are novel chiral alcohols for use as chiral building blocks for the synthesis of more complex enantiomerically enriched molecules. The synthesis, isolation and characterization of both enantiomers of the structurally unique chiral secondary alcohol 2-methyl-(2,5-dimethylphenyl)propanol, (R)-2-methyl-(2,5-dimethylphenyl)propanol and (S)-2-methyl-(2,5-dimethylphenyl)propanol, is disclosed herein. Also provided are the methods of preparing the stereoisomers, and compositions containing them.

### DETAILED DESCRIPTION OF THE INVENTION

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

### 1. Definitions

It must be noted that as used herein and in the appended claims, the singular forms “a,” “an,” and “the” include plural references unless the context clearly dictates otherwise.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety. Nothing herein is to be construed as an admission that the invention is not entitled to antedate such disclosure by virtue of prior invention.

“Optically active” is intended to mean an enantiomerically enriched sample or a sample with a detectable “enantiomeric excess.” “Enantiomeric excess” or “ee,” is the excess of one of two enantiomers over the other, usually expressed as a percentage, i.e., a 90% ee reflects the presence of 95% of one enantiomer and 5% of the other in the material in question.

The term “chiral reducing agent” is intended to mean a chiral reagent, or combination of reagents, capable of converting a ketone to the corresponding (R)- or (S)-alcohol. The chiral reducing agent is therefore either “(R)-producing” or “(S)-producing,” and is selected based on its ability to produce the desired enantiomer of a compound of formula I. An “(R)-producing” chiral reagent is a chiral reagent that, upon reduction of the ketone, produces the (R)-enantiomer alcohol. An “(S)-producing” chiral reagent is a chiral reagent that, upon reduction of the ketone, produces the (S)-enantiomer alcohol. The chiral reducing agent can be, for example, a chiral hydride reagent, such as B-chlorodiisopinocampheylborane, B-methoxydiisopinocampheylborane, NB-enantride™ (9-BBN-nopol benzyl ether adduct) and Alpine Borane® (B-isopinocampheyl-9-borabicyclo[3.3.1]nonane) or other organoboranes, for example. Alternatively, various catalysts are employed in conjunction with a hydride reagent for the conversion of ketones to the corresponding alcohol, such as CBS-catalyst. “CBS catalyst” is intended to mean 2-methyl-CBS-oxazaborolidine or 1 -methyl-3,3-diphenylhexahydropyrrolo[1,2-c][1,3,2]oxazaborole.

Various oxazaborolidine catalysts can be substituted in the methods of this invention. Examples of such reagents are shown below as compounds of formula II:

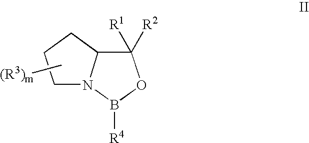

wherein;

R¹ and R² are independently selected from hydrogen, C1-C6 alkyl, C1-C6 substituted alkyl, aryl, and substituted aryl, or R¹ and R² taken together are oxo;

R³ is independently selected from hydrogen, C1-C6 alkyl, C1-C6 substituted alkyl, aryl, and substituted aryl;

R⁴ is selected from hydrogen, C1-C10 alkyl, C1-C10 substituted alkyl, aryl, substituted aryl heteroaryl, and substituted heteroaryl; and

m is 0 to 7, and stereoisomers thereof.

Examples of other oxazaborolidines include the tricyclic and bicyclic oxazaborolidines in Scheme 1. Only one enantiomer of each oxazaborolidine catalyst is shown. The enantiomer of each structure delineated in Scheme 1 is implied, since these catalysts would be a route to the other enantiomer of the alcohol derived from stereoselective reduction of the ketone.

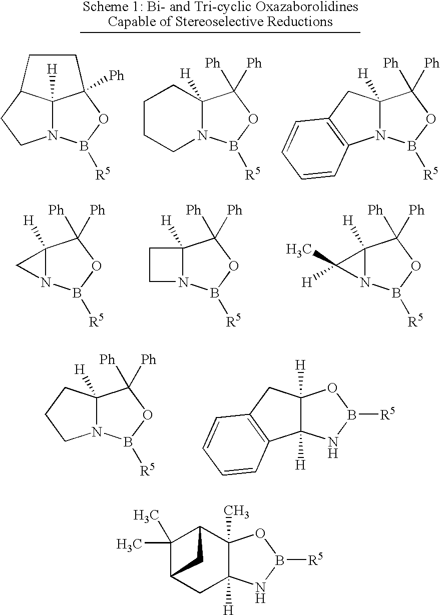

wherein “Ph” refers to phenyl and R⁵ is

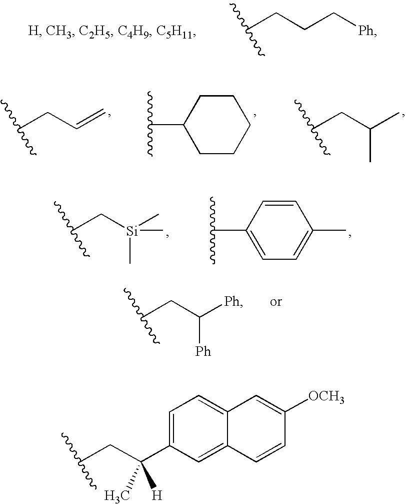

wherein the wavy line indicates the point of attachment to the remainder of the molecule.

As used herein, the term “solvent” is intended to mean a liquid which contains at least one substance capable of bringing at least one other substance into solution. Thus, the solvent might be at least one organic and/or inorganic solvent or can alternatively be an alkaline or acid solution. Examples of “organic solvents” include, but are not limited to, tetrahydrofuran, diethyl ether, methyl tert-butyl ether, dioxane, hexane, pentane, benzene, toluene, methylene chloride and combinations thereof. “Inorganic solvents” include, but are not limited to, water or aqueous solutions of inorganic salts as well as molten salts and liquid ammonia, liquid sulfur dioxide, dinitrogen tetroxide, antimony trichloride, bromine pentafluoride, hydrogen fluoride, pure sulphuric acid, other inorganic acids and combinations thereof. In addition, ionic liquids are included by the scope of the reactions disclosed herein. Examples of ionic liquids include, but are not limited to, those derived from methylimidazolium and pyridinium ions, such as 1-butyl-3-methylimidazolium tetrafluoroborate and 1-butyl-4-methylpyridinium hexafluorophosphate, and the like as well as combinations thereof. Further, the solvent is selected such that it does not react with, or is inert toward, the compounds of the invention.

The term “hydride reagent” is intended to mean a reagent capable of delivering a hydride, for example, borane methylsulfide complex ((CH₃)₂SBH₃). Suitable hydride reagents for inclusion in the methods of this invention include other borohydride reagents such as lithium aluminum hydride, lithium borohydride, sodium borohydride, sodium triacetoxyborohydride, sodium cyanoborohydride, potassium tri-sec-butylborohydride, catecholborane, and boranes complexed with amines and other heteroatom-bearing Lewis bases such as tetrabutylammonium borohydride, lithium dimethylaminoborohydride and the like.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃₋), ethyl (CH₃CH₂₋), n-propyl (CH₃CH₂CH₂—), isopropyl ((CH₃)₂CH—), n-butyl (CH₃CH₂CH₂CH₂₋), isobutyl ((CH₃)₂CHCH₂—), sec-butyl ((CH₃)(CH₃CH₂)CH—), n-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5 hydrogens replaced with substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, sulfonylamino, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, sulfonyl, sulfonyloxy, thioacyl, thiol, alkylthio, and substituted alkylthio, wherein said substituents are defined herein. In some implementations, the alkyl has 1 to 3 of the aforementioned groups. In other implementations, the alkyl has 1 to 2 of the aforementioned groups.

“Alkylene” refers to divalent saturated aliphatic hydrocarbyl groups preferably having from 1 to 6 and more preferably 1 to 3 carbon atoms that are either straight-chained or branched. This term is exemplified by groups such as methylene (—CH₂₋), ethylene (—CH₂CH₂₋), n-propylene (—CH₂CH₂CH₂₋), iso-propylene (—CH₂CH(CH₃)—) or (—CH(CH₃)CH₂—), and the like.

“Substituted alkylene” refers to an alkylene group having from 1 to 3 hydrogens replaced with substituents selected from the group consisting of alkyl, substituted alkyl, alkoxy, substituted alkoxy, acyl, acylamino, acyloxy, amino, substituted amino, aminocarbonyl, aryl, substituted aryl, aryloxy, substituted aryloxy, cyano, halogen, hydroxyl, nitro, carboxyl, carboxyl ester, cycloalkyl, substituted cycloalkyl, heteroaryl, substituted heteroaryl, heterocyclic, substituted heterocyclic, and oxo wherein said substituents are defined herein. In some implementations, the alkylene has 1 to 2 of the aforementioned groups. It is to be noted that when the alkylene is substituted by an oxo group, 2 hydrogens attached to the same carbon of the alkylene group are replaced by “═O”.

“Alkoxy” refers to the group —O-alkyl, wherein alkyl is as defined herein. Alkoxy includes methoxy, ethoxy, n-propoxy, isopropoxy, n-butoxy, t-butoxy, sec-butoxy, n-pentoxy, and the like.

“Substituted alkoxy” refers to the group —O-(substituted alkyl), wherein substituted alkyl is as defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)-cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, where one of R²¹ and R²² is sulfonyl, and wherein R²¹ and R²² are optionally joined together with the nitrogen bound thereto to form a heterocyclic or substituted heterocyclic group, provided that R²¹ and R²² are not both hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, sulfonyl, substituted heteroaryl, heterocyclic, and substituted heterocyclic are as defined herein. When R²¹ is hydrogen and R²² is alkyl, the substituted amino group is sometimes referred to herein as “alkylamino.” When R²¹ and R²² are alkyl, the substituted amino group is sometimes referred to herein as “dialkylamino.” When referring to a monosubstituted amino, it is meant that either R²¹ or R²² is hydrogen, but not both. When referring to a disubstituted amino, it is meant that neither R²¹ nor R²² is hydrogen.

“Aminocarbonyl” refers to the group —C(O)NR²¹ R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²⁰C(O)NR²¹R²², wherein R²⁰ is hydrogen or alkyl and R²¹and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminothiocarbonylamino” refers to the group —NR²⁰C(S)NR²¹R²², wherein R²⁰ is hydrogen or alkyl and R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R ²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminocarbonyloxy” refers to the group —O—C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonyl” refers to the group —SO₂ NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonyloxy” refers to the group —O—SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic; R²¹ and R²² are optionally joined together with the nitrogen bound thereto to form a heterocyclic or substituted heterocyclic group; and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR²⁰—SO₂NR²¹R²², wherein R²⁰ is hydrogen or alkyl and R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR³⁰)NR³¹R³², wherein R³¹ and R³² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R³¹ and R³² are optionally joined together with the nitrogen bound thereto to form a heterocyclic or substituted heterocyclic group. R³⁰ is selected from the group consisting of hydrogen, alkyl, substituted alkyl, alkenyl, substituted alkenyl, alkynyl, substituted alkynyl, cycloalkynyl, substituted cycloalkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, nitro, nitroso, hydroxy, alkoxy, cyano, acyl, —SO₂-alkyl and —SO₂-substituted alkyl, wherein alkyl, substituted alkyl, alkenyl, substituted alkenyl, alkynyl, substituted alkynyl, cycloalkynyl, substituted cycloalkynyl, cycloalkyl, substituted cycloalkyl, cycloalkenyl, substituted cycloalkenyl, aryl, substituted aryl, heteroaryl, substituted heteroaryl, heterocyclic, substituted heterocyclic, nitro, nitroso, hydroxy, alkoxy, and cyano are as defined herein.

“Aryl” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic provided that the point of attachment is through an atom of the aromatic aryl group. For example, 1,2,3,4-tetrahydronaphthalen-5-yl, 9H-fluoren-2-yl, and the like. Preferred aryl groups include phenyl and naphthyl.

“Substituted aryl” refers to aryl groups having 1 to 5 hydrogens replaced with substituents independently selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, —SO₃H, sulfonyl, sulfonyloxy, thioacyl, thiol, alkylthio, and substituted alkylthio, wherein said substituents are as defined herein. In some implementations, the aryl has 1 to 3 of the aforementioned groups. In other implementations, the aryl has 1 to 2 of the aforementioned groups. In some implementations, substituted aryl includes compounds containing oxo substituent in the non-aromatic ring fused to the aryl group. For example, 1-oxo-indan-4-yl, wherein the point of attachment is through the phenyl ring.

“Aryloxy” refers to the group —O-aryl, wherein aryl is as defined herein, including, by way of example, phenoxy, naphthoxy, and the like.

“Substituted aryloxy” refers to the group —O-(substituted aryl), wherein substituted aryl is as defined herein.

“Arylthio” refers to the group —S-aryl, wherein aryl is as defined herein. In other implementations, sulfur may be oxidized to —S(O)— or —SO₂— moieties. Sulfoxides may exist as one or more stereoisomers, e.g. methylsulfinylethane is a chiral molecule having two enantiomeric forms, R and S.

“Substituted arylthio” refers to the group —S-(substituted aryl), wherein substituted aryl is as defined herein. In other implementations, sulfur may be oxidized to —S(O)— or —SO₂— moieties. The sulfoxide may exist as one or more stereoisomers.

“Alkenyl” refers to straight chain or branched hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified by vinyl, allyl, and but-3-en-1-yl. Included within this term are the cis and trans isomers or mixtures of these isomers.

“Substituted alkenyl” refers to alkenyl groups having from 1 to 3 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, sulfonyl, sulfonyloxy, thioacyl, thiol, alkylthio, and substituted alkylthio, wherein said substituents are as defined herein and with the proviso that any hydroxy substitution is not attached to a vinyl (unsaturated) carbon atom. In some implementation s, the alkenyl has 1 to 2 of the aforementioned groups.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic —CC— unsaturation. Examples of such alkynyl groups include acetylenyl (—CCH), and propargyl (—CH₂CCH).

“Substituted alkynyl” refers to alkynyl groups having from 1 to 3 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, sulfonyl, sulfonyloxy, thioacyl, thiol, alkylthio, and substituted alkylthio, wherein said substituents are as defined herein and with the proviso that any hydroxy or thiol substitution is not attached to an acetylenic carbon atom. In some implementations, the alkynyl has 1 to 2 of the aforementioned groups.

“Alkynyloxy” refers to the group —O-alkynyl, wherein alkynyl is as defined herein. Alkynyloxy includes, by way of example, ethynyloxy, propynyloxy, and the like.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the groups —NR—C(O)O-alkyl, —NR—C(O)O-substituted alkyl, —NR—C(O)O-alkenyl, —NR—C(O)O-substituted alkenyl, —NR—C(O)O-alkynyl, —NR—C(O)O-substituted alkynyl, —NR—C(O)O-aryl, —NR—C(O)O-substituted aryl, —NR—C(O)O-cycloalkyl, —NR—C(O)O-substituted cycloalkyl, —NR—C(O)O-cycloalkenyl, —NR—C(O)O-substituted cycloalkenyl, —NR—C(O)O-heteroaryl, —NR—C(O)O-substituted heteroaryl, —NR—C(O)O-heterocyclic, and —NR—C(O)O-substituted heterocyclic, wherein R is alkyl or hydrogen and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the groups —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 13 carbon atoms having single or multiple cyclic rings including fused, bridged, and spiro ring systems. Examples of cycloalkyl groups include adamantyl, cyclopropyl, cyclobutyl, cyclopentyl, cyclooctyl, and the like. One or more rings fused to the cycloalkyl group can be aromatic, provided that the point of attachment is through the non-aromatic ring, e.g. 9H-fluoren-9-yl,1,2,3,4-tetrahydronaphthalen-2-yl, and the like.

“Cycloalkenyl” refers to non-aromatic cyclic alkyl groups of from 3 to 10 carbon atoms having single or multiple rings and having at least one double bond and preferably from 1 to 2 double bonds.

“Cycloalkynyl” refers to non-aromatic cycloalkyl groups of from 7 to 12 carbon atoms having single or multiple rings and having at least one triple bond.

“Cycloalkylene” refers to divalent cycloalkyl groups, wherein cycloalkyl is as defined herein.

“Substituted cycloalkylene” refers to cycloalkylene group having from 1 to 3 hydrogens replaced with substituents selected from the group consisting of alkyl, substituted alkyl, alkoxy, substituted alkoxy, acyl, acylamino, acyloxy, amino, substituted amino, aminocarbonyl, aryl, substituted aryl, aryloxy, substituted aryloxy, cyano, halogen, hydroxyl, nitro, carboxyl, carboxyl ester, cycloalkyl, substituted cycloalkyl, heteroaryl, substituted heteroaryl, heterocyclic, substituted heterocyclic, and oxo wherein said substituents are as defined herein. In some implementations, the alkylene has 1 to 2 of the aforementioned groups. It is to be noted that when the cycloalkylene is substituted by an oxo group, 2 hydrogens attached to the same carbon of the cycloalkylene group are replaced by “═O”.

“Substituted cycloalkyl,” “substituted cycloalkenyl,” and “substituted cycloalkynyl” refer to a cycloalkyl, cycloalkenyl, or cycloalkynyl group having from 1 to 5 substituents selected from the group consisting of oxo, thioxo, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, —SO₃H, sulfonyl, sulfonyloxy, thioacyl, thiol, alkylthio, and substituted alkylthio, wherein said substituents are as defined herein, provides that any hydroxy or thiol substitution is not attached to an unsaturated carbon atom. In some implementations, the cycloalkyl or cycloalkenyl has 1 to 3 of the aforementioned groups.

“Cycloalkoxy” refers to —O-cycloalkyl.

“Substituted cycloalkoxy” refers to —O-(substituted cycloalkyl).

“Cycloalkylthio” refers to —S-cycloalkyl. In other implementations, sulfur may be oxidized to —S(O)— or —SO₂— moieties. The sulfoxide may exist as one or more stereoisomers.

“Substituted cycloalkylthio” refers to —S-(substituted cycloalkyl). In other implementations, sulfur may be oxidized to —S(O)—, or —SO₂— moieties. The sulfoxide may exist as one or more stereoisomers.

“Cycloalkenyloxy” refers to —O-cycloalkenyl.

“Substituted cycloalkenyloxy” refers to —O-(substituted cycloalkenyl).

“Cycloalkenylthio” refers to —S-cycloalkenyl. In other implementations, sulfur may be oxidized to sulfinyl or sulfonyl moieties. The sulfoxide may exist as one or more stereoisomers.

“Substituted cycloalkenylthio” refers to —S-(substituted cycloalkenyl). In other implementations, sulfur may be oxidized to —S(O)— or —SO₂— moieties. The sulfoxide may exist as one or more stereoisomers.

“Guanidino” refers to the group —NHC(═NH)NH₂.

“Substituted guanidino” refers to the group —NR³³ C(═NR³³)N(R³³)₂, wherein each R³³ independently is selected from the group consisting of hydrogen, alkyl, substituted alkyl, aryl, substituted aryl, heteroaryl, substituted heteroaryl, heterocyclic, and substituted heterocyclic; two R groups attached to a common guanidino nitrogen atom are optionally joined together with the nitrogen bound thereto to form a heterocyclic or substituted heterocyclic group, provided that at least one R is not hydrogen; and said substituents are as defined herein.

“Halo” or “halogen” refers to fluoro, chloro, bromo, and iodo and is preferably fluoro or chloro.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen, and sulfur within the ring. Such heteroaryl groups can have a single ring (e.g., pyridinyl or furyl) or multiple condensed rings (e.g., indolizinyl or benzothienyl), wherein the condensed rings may or may not be aromatic and/or contain a heteroatom, provided that the point of attachment is through an atom of the aromatic group containing the heteroatom. In one implementation, the nitrogen and/or sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5 substituents selected from the group consisting of the same group of substituents defined for substituted aryl. In some implementations, the heteroaryl has 1 to 3 of the aforementioned groups. In other implementations, the heteroaryl has 1 to 2 of the aforementioned groups.

“Heteroaryloxy” refers to —O-heteroaryl.

“Substituted heteroaryloxy” refers to the group —O-(substituted heteroaryl).

“Heteroarylthio” refers to the group —S-heteroaryl. In other implementations, sulfur may be oxidized to —S(O)— or —SO₂— moieties. The sulfoxide may exist as one or more stereoisomers.

“Substituted heteroarylthio” refers to the group —S-(substituted heteroaryl). In other implementations, sulfur may be oxidized to —S(O)— or —SO₂— moieties. The sulfoxide may exist as one or more stereoisomers.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 15 ring atoms, including 1 to 4 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In one implementation, the nitrogen and/or sulfur atom(s) of the heterocyclic group are optionally oxidized to provide for the N-oxide, —S(O)—, or —SO₂— moieties.

“Substituted heterocyclic,” “substituted heterocycloalkyl,” and “substituted heterocyclyl” refer to heterocyclyl groups that are substituted with from 1 to 5 of the same substituents as defined for substituted cycloalkyl. In some implementations, the heterocyclyl has 1 to 3 of the aforementioned groups.

“Heterocyclyloxy” refers to the group —O-heterocyclyl.

“Substituted heterocyclyloxy” refers to the group —O-(substituted heterocyclyl).

“Heterocyclylthio” refers to the group —S-heterocyclyl. In other implementations, sulfur may be oxidized to —S(O)— or —SO₂— moieties. The sulfoxide may exist as one or more stereoisomers.

“Substituted heterocyclylthio” refers to the group —S-(substituted heterocyclyl). In other implementations, sulfur may be oxidized to —S(O)— or —SO₂— moieties. The sulfoxide may exist as one or more stereoisomers.

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

“Nitro” refers to the group —NO₂.

“Nitroso” refers to the group —NO.

“Oxo” refers to the atom (═O).

“Sulfonyl” refers to the group —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, and —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cycloalkyl, —OSO₂-cycloalkenyl, —OSO₂-substituted cylcoalkenyl, —OSO₂-aryl, —OSO₂-substituted aryl, —OSO₂-heteroaryl, —OSO₂-substituted heteroaryl, —OSO₂-heterocyclic, and —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thioxo” refers to the atom (═S).

“Alkylthio” refers to the group —S-alkyl, wherein alkyl is as defined herein. In other implementations, sulfur may be oxidized to —S(O)—. The sulfoxide may exist as one or more stereoisomers.

“Substituted alkylthio” refers to the group —S-(substituted alkyl), wherein substituted alkyl is as defined herein. In other implementations, sulfur may be oxidized to —S(O)—. The sulfoxide may exist as one or more stereoisomers.

“Stereoisomer” and “stereoisomers” refer to compounds that have same atomic connectivity but different atomic arrangement in space. Stereoisomers include cis-trans isomers, E and Z isomers, enantiomers, and diastereomers.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that, for example, substituted aryl groups are limited to—substituted aryl-(substituted aryl)-substituted aryl.

Similarly, it is understood that the above definitions are not intended to include impermissible substitution patterns (e.g., methyl substituted with 5 fluoro groups or a hydroxy group alpha to ethenylic or acetylenic unsaturation). Such impermissible substitution patterns are well known to the skilled artisan.

### 2. Compositions

Both the (R)- and (S)-enantiomers of 2-methyl(2,5-dimethylphenyl)propanol are disclosed herein (formula I).

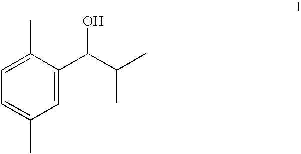

In one aspect, the present invention discloses 2-methyl-(2,5-dimethylphenyl)propanol comprising predominantly the (S)-enantiomer and a residual amount of the (R)-enantiomer, wherein the (S)-enantiomer is present in an enantiomeric excess of at least about 95% over the (R)-enantiomer. In another aspect, the (S)-enantiomer is present in an enantiomeric excess of greater than about 99%, or alternatively, greater than about 90%, or alternatively, greater than about 80%, or alternatively, greater than about 70%, or alternatively, greater than about 50%, or alternatively, greater than about 10%.

Another aspect of the present invention discloses 2-methyl-(2,5-dimethylphenyl)propanol comprising predominantly the (R)-enantiomer and a residual amount of the (S)-enantiomer, wherein the (R)-enantiomer is present in an enantiomeric excess of at least about 95% over the (S)-enantiomer. Alternatively the (R)-enantiomer is present in an enantiomeric excess of greater than about 99%, or alternatively, greater than about 90%, or alternatively, greater than about 80%, or alternatively, greater than about 70%, or alternatively, greater than about 50%, or alternatively, greater than about 10%.

In one embodiment, the compounds or compositions (indicating the presence of a small amount of the other enantiomer) are provided in carriers. These carriers include organic solvents, inorganic solvents and mixtures thereof. These solvents are described in the definitions section.

In yet another aspect, the present invention discloses (S)-2-methyl-(2,5-dimethylphenyl)propanol. In yet another aspect, the present invention discloses (R)-2-methyl-(2,5-dimethylphenyl)propanol. Both of these optically active alcohols are useful as chiral building blocks in chiral synthesis. In addition, they could be utilized as chiral auxiliaries or used as ligands or ligand precursors in organometallic catalysts or as alcohol precursors to other types of ligands, such as thiols or amines.

### 3. Methods of the Inventions

Also provided are methods of making (S)-2-methyl-(2,5-dimethylphenyl)propanol and (R)-2-methyl-(2,5-dimethylphenyl)propanol. The compounds may be made by contacting 2-methyl-(2,5-dimethylphenyl)propanone with a chiral reducing agent under suitable reaction conditions.

In another aspect, the present invention discloses (S)-2-methyl-(2,5-dimethylphenyl)propanol formed by a process comprising reacting 2-methyl-(2,5-dimethylphenyl)propanone with a chiral reducing agent. In yet another aspect, the present invention discloses (R)-2-methyl-(2,5-dimethylphenyl)propanol formed by a process comprising reacting 2-methyl-(2,5-dimethylphenyl)propanone with a chiral reducing agent.

In yet another aspect, the present invention discloses a method of isolating (S)-2-methyl-(2,5-dimethylphenyl)propanol. In addition, the present invention discloses a method of isolating (R)-2-methyl-(2,5-dimethylphenyl)propanol.

In another aspect, the present invention discloses (S)-2-methyl-(2,5-dimethylphenyl)propanol formed by a process comprising reacting 2-methyl-(2,5-dimethylphenyl)propanone with the (R)-CBS-catalyst and suitable hydride reagent, such as borane methylsulfide complex. Alternatively, the present invention discloses (R)-2-methyl-(2,5-dimethylphenyl)propanol formed by a process comprising reacting 2-methyl-(2,5-dimethylphenyl)propanone with the (S)-CBS-catalyst and suitable hydride reagent, such as borane methylsulfide complex. Reagents such as borane tetrahydrofuran complex, lithium borohydride, sodium borohydride, sodium triacetoxyborohydride, catecholborane, catecholborane-amine complexes, and the like can be substituted for borane methylsulfide complex in the present invention. Various oxazaborolidine catalysts can be substituted in the methods of this invention. Examples of such reagents are shown below in formula II:

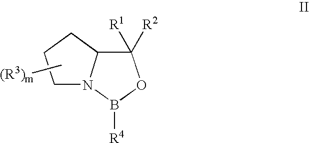

wherein;

R¹ and R² are independently selected from hydrogen, C1-C6 alkyl, C1-C6 substituted alkyl, aryl, and substituted aryl, or R¹ and R² taken together are oxo;

R³ is independently selected from hydrogen, C1-C6 alkyl, C1-C6 substituted alkyl, aryl, and substituted aryl;

R⁴ is selected from hydrogen, C1-C10 alkyl, C1-C10 substituted alkyl, aryl, substituted aryl heteroaryl, and substituted heteroaryl; and

m is 0 to 7, and stereoisomers thereof.

Such chiral oxazaborolidine catalysts are available from commercial sources or can be easily obtained by known methods. For example, an alkyl or aryl boronic acid can be reacted with proline or a proline derivative under water excluding conditions (Dean-Stark apparatus, calcium hydride, molecular sieves or the like) and used as a solution without prior isolation.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

In these examples and elsewhere, abbreviations have the following meanings:
d=doublet
ee=enantiomeric excess
g=gram
Hz=hertz
J=splitting
M=molar
MHz=megahertz
mL=milliliter
mmol=millimole
NMR=nuclear magnetic resonance
s=singlet
THF=tetrahydrofuran
δ=chemical shift in ppm (parts per million)

### Example 1

### CBS-Catalyzed Reduction of 2-methyl-(2,5-dimethylphenyl)propanone

To a dry round bottom flask under nitrogen was added 2-methyl-(2,5-dimethylphenyl)propanone (1 mL, approx. 1 g, 6.4 mmol), followed by dry THF (10 mL), and finally CBS catalyst (0.178 g, 0.64 mmol). The solution was then cooled on an ice bath to zero degrees Celsius. To the cooled solution was added dropwise by syringe (CH₃)₂SBH₃ (0.061 mL of a 1M solution, 0.64 mmol). The reaction was left to stir for one hour followed by another addition of (0.061 mL, 0.64 mmol). This was repeated two more times on the hour completing the addition (CH₃)₂SBH₃ (0.245 mL, 2.56 mmol total). The solution was left to stir until no more reactant was detected by NMR (approximately 1.5 hours). The solution was then combined with three times volume of water (30 mL) and extracted with chloroform (3×10 mL). The organic fraction was washed with saturated sodium bicarbonate and brine, concentrated under vacuum and purified on a silica column (10/90 ethyl acetate/hexane). Proton NMR data (in CDCl₃, 400 MHz): δ 7.206 (s, 1H), 6.972 (m, 2H), 4.571 (d, J=6.8 Hz, 1H), 2.300 (s, 3H), 2.265 (d, J=6.8 Hz, 1H), 1.942 (d, J=6.8 Hz, 1H), 1.713 (s, 1H), 1.023 (d, J=6.8 Hz, 3H), 0.815 (d, J=6.8 Hz, 3H).

The determination of enantiomeric excess can be accomplished by various methods known to those in the art. For example, chiral high-pressure liquid chromatography, chiral gas liquid chromatography and NMR with the use of chiral shift reagents, and the like.

What is claimed is:

1. A composition comprising a compound of formula I: 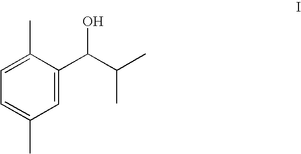
wherein the (S)-enantiomer is present in an enantiomeric excess of at least about 95% over the (R)-enantiomer.

2. The composition of claim 1, further comprising a carrier.

3. The composition of claim 2, wherein the carrier is selected from the group consisting of an organic solvent, an inorganic solvent, and a combination thereof.

4. A method of preparing a composition of claim 1, comprising contacting 2-methyl-(2,5-dimethylphenyl)propanone with an (S)-producing chiral reducing agent under reaction conditions to form the compound of formula I.

5. The method of claim 4, wherein the chiral reducing agent is a hydride reagent in the presence of a (R)-CBS-catalyst.

6. The method of claim 4 further comprising isolating the (S)-enantiomer.

7. A composition comprising compound of formula I: 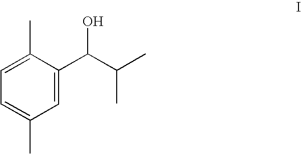
wherein the (R)-enantiomer is present in an enantiomeric excess of at least about 95% over the (S)-enantiomer.

8. The composition of claim 7 further comprising a carrier.

9. The composition of claim 8, wherein the carrier is selected from the group consisting of an organic solvent, an inorganic solvent, and combinations thereof.

10. A method of preparing a composition of claim 7, comprising reacting 2-methyl-(2,5-dimethylphenyl)propanone with an (R)-producing chiral reducing agent under reaction conditions to form the compound of formula I.

11. The method of claim 10, wherein the chiral reducing agent is a hydride reagent in the presence of a (S)-CBS-catalyst.

12. The method of claim 6 further comprising isolating the compound of formula I.

13. The method of either claim 4 or 10, wherein the chiral reducing agent comprises a compound of formula II: 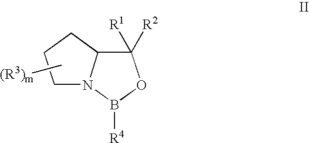
wherein;
R¹ and R² are independently selected from hydrogen, C1-C6 alkyl, C1-C6 substituted alkyl, aryl, and substituted aryl, or R¹ and R² taken together are oxo;
R³ is independently selected from hydrogen, C1-C6 alkyl, C1-C6 substituted alkyl, aryl, and substituted aryl;
R⁴ is selected from hydrogen, C1-C10 alkyl, C1-C10 substituted alkyl, aryl, substituted aryl heteroaryl, and substituted heteroaryl; and
m is 0 to 7, and stereoisomers thereof.

14. Optically active (S)-2-methyl-(2,5-dimethylphenyl)propanol.

15. Optically active (R)-2-methyl-(2,5-dimethylphenyl)propanol.
